SEQUENCE LISTING (24 sequences)

SEQ ID NO: 1 - DNA, 91 bp, Artificial Sequence
  probe
gcttgtcttcgagtaaggttaaaaacgcattagcgtcatcattctaaactacgctaatgcgtttttaaccttactcgaagacaagcttttt

SEQ ID NO: 2 - DNA, 91 bp, Artificial Sequence
  probe
aaagtctgcttaacgccattgcgagctatgttcgctcatcattcattactagcgaacatagctcgcaatggcgttaagcagactttttttt

SEQ ID NO: 3 - DNA, 91 bp, Artificial Sequence
  probe
aaaccaggagtaggattctcttttagaaaagaatttcattcatactactaaaattcttttctaaaagagaatcctactcctggtttttttt

SEQ ID NO: 4 - DNA, 91 bp, Artificial Sequence
  probe
cgcgtgcagtcttatcgaattgttcttcccgggtatcatctaactaactaatacccgggaagaacaattcgataagactgcacgcgttttt

SEQ ID NO: 5 - DNA, 91 bp, Artificial Sequence
  probe
ctccggatccaccgtttcaggggctttattttctatcatctaacattactatagaaaataaagcccctgaaacggtggatccggagttttt

SEQ ID NO: 6 - DNA, 91 bp, Artificial Sequence
  probe
gcttaagtcatagcgtttccattctttattaaagatcatctaacattctaatctttaataaagaatggaaacgctatgacttaagcttttt

SEQ ID NO: 7 - DNA, 91 bp, Artificial Sequence
  probe
tcaattctggaaatggtgttaaacccggaaaatcgtcatactaactactaacgattttccgggtttaacaccatttccagaattgattttt

SEQ ID NO: 8 - DNA, 91 bp, Artificial Sequence
  probe
acactgatgttcatttgttagtctctttttacaatactactaatcatcattattgtaaaaagagactaacaaatgaacatcagtgtttttt

SEQ ID NO: 9 - DNA, 16 bp, Artificial Sequence
  Internal control probe
tagtttagaatgatga

SEQ ID NO: 10 - DNA, 16 bp, Artificial Sequence
  Internal control probe
tagtaatgaatgatga

SEQ ID NO: 11 - DNA, 16 bp, Artificial Sequence
  Internal control probe
ttagtagtatgaatga

SEQ ID NO: 12 - DNA, 16 bp, Artificial Sequence
  Internal control probe
ttagttagttagatga

SEQ ID NO: 13 - DNA, 16 bp, Artificial Sequence
  Internal control probe
tagtaatgttagatga

SEQ ID NO: 14 - DNA, 16 bp, Artificial Sequence
  Internal control probe
ttagaatgttagatga

SEQ ID NO: 15 - DNA, 16 bp, Artificial Sequence
  Internal control probe
ttagtagttagtatga

SEQ ID NO: 16 - DNA, 16 bp, Artificial Sequence
  Internal control probe
actactaatcatcatt

SEQ ID NO: 17 - DNA, 35 bp, Artificial Sequence
  Reporter probe
gcttgtcttcgagtaaggttaaaaacgcattagcg

SEQ ID NO: 18 - DNA, 35 bp, Artificial Sequence
  Reporter probe
aaagtctgcttaacgccattgcgagctatgttcgc

SEQ ID NO: 19 - DNA, 35 bp, Artificial Sequence
  Reporter probe
aaaccaggagtaggattctcttttagaaaagaatt

SEQ ID NO: 20 - DNA, 35 bp, Artificial Sequence
  Reporter probe
cgcgtgcagtcttatcgaattgttcttcccgggta

SEQ ID NO: 21 - DNA, 35 bp, Artificial Sequence
  Reporter probe
ctccggatccaccgtttcaggggctttattttcta

SEQ ID NO: 22 - DNA, 35 bp, Artificial Sequence
  Reporter probe
gcttaagtcatagcgtttccattctttattaaaga

SEQ ID NO: 23 - DNA, 35 bp, Artificial Sequence
  Reporter probe
tcaattctggaaatggtgttaaacccggaaaatcg

SEQ ID NO: 24 - DNA, 35 bp, Artificial Sequence
  Reporter probe
acactgatgttcatttgttagtctctttttacaat

(19) United States
(12) Patent Application Publication
(10) Pub. No.: US 2014/0087372 A1
(43) Pub. Date: Mar. 27, 2014

(54) PROBE, KIT, AND METHOD FOR DETECTING PATHOGENIC MICROORGANISM

(71) Applicant: CHUNG YUAN CHRISTIAN UNIVERSITY, Jhongli City (TW)

(72) Inventors: Chung-Yung CHEN, Jhongli City (TW); Tzong-Yuan WU, New Taipei City (TW)

(73) Assignee: CHUNG YUAN CHRISTIAN UNIVERSITY, Jhongli City (TW)

(21) Appl. No.: 13/628,859

(22) Filed: Sep. 27, 2012

(51) Int. Cl. C07H 21/04; C40B 40/06; G01N 21/64; B82Y 15/00

(52) U.S. Cl. 435/6.11; 536/24.32; 506/16; 977/774; 977/920

(57) ABSTRACT

Disclosed herein is a hairpin probe for detecting a target pathogenic microorganism in a sample. The hairpin probe includes a microbead and an oligonucleotide having its 3′-end coupled to the microbead. The oligonucleotide includes, from 5′ to 3′, a Tag sequence hybridizable to a specific identification sequence of the pathogenic microorganism, an internal control sequence having at least four words each having 4 nucleotides with a 75% AT-content, an anti-Tag sequence being a reverse complement of the Tag sequence, and a tail having at least two consecutive thymidine residues. The Tag and anti-Tag sequences are operable to form a stem of the hairpin probe with the internal control sequence being a loop. Also disclosed herein are, a kit including the hairpin probe and a method for using the kit in the detection of a target pathogenic microorganism.

### BACKGROUND OF THE INVENTION

1. Field of the Invention

The present disclosure relates to the field of pathogen detection. More particularly, the disclosure relates to a hairpin probe, kit, and method for qualitatively and quantitatively detecting at least one target pathogenic microorganism in a sample.

2. Description of Related Art

Infectious diseases are the leading cause of mortality. The detection of the pathogenic microorganism responsible for the diseases is thus of great importance for diagnosis, therapy, and control of the disease.

The predominant techniques currently used to identify pathogenic microorganisms rely upon culture and susceptibility tests, PCR-based methods, and immunological methods. However, these approaches suffer from a number of considerable drawbacks. Standard culture and susceptibility tests are laborious and time-consuming that typically take days even weeks to identify a pathogen.

Disadvantages of PCR-based methods of include their relative complexity, high false-positive rate, and high cost. Limitations of Immunological methods include low sensitivity and variable specificity.

In the fields of water and environment quality control, clinical diagnosis, and food industry, the complexity of samples presents more challenges to pathogen detection. For example, the presence of potentially interfering material in environmental, manufacturing (e.g., food processing), and clinical samples vary widely and may lead to false positives, false negatives, or reduced accuracy and sensitivity of biological assays.

Concerns over the accuracy, speed of analysis, and the limited ability of these techniques to detect multiple strains or pathogens have prompted researchers to consider alternative detection platforms.

In view of the foregoing, there exist problems and disadvantages in the current pathogen detection techniques that await further improvement. Accordingly, there is an urgent need in the related art for providing a rapid, sensitive, and accurate tool for pathogen detection.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a hairpin probe for detecting a target pathogenic microorganism in a sample. The hairpin probe has a stem-and-loop structure and comprises a microbead and an oligonucleotide having its 3′-end coupled to the microbead. According to principles and spirits of the present invention, the oligonucleotide is designed in such a way that it may specifically hybridize with a specific identification sequence of the target pathogenic microorganism. Also, the oligonucleotide, in conjunction with conjugates according to another aspect/embodiment of the present disclosure, is operable to provide qualitative and quantitative analysis of the pathogenic microorganism.

According to one embodiment of the present disclosure, the oligonucleotide of the hairpin probe comprises, from 5′ to 3′, a Tag sequence, an internal control sequence, an anti-Tag sequence, and a tail. The Tag sequence is hybridizable to a specific identification sequence of the target pathogenic microorganism. The internal control sequence comprises at least four words, wherein each word consists of 4 nucleotides with a 75% AT-content, wherein the internal control sequence is operable to form the loop. The anti-Tag sequence is a reverse complement of the Tag sequence so that the anti-Tag sequence and Tag sequence are operable to form the stem. The tail comprises at least two consecutive thymidine residues.

According to various embodiments of the present disclosure, the microbead comprises a material selected from the group consisting of polystyrene, crosslinked poly(styrene/divinylbenzene), poly(methyl methacrylate), and melamine. Generally, the microbead has a diameter of about 1-50 μm; preferably, about 5-8.5 μm.

In certain embodiments of the present invention, the Tag sequence and the anti-Tag sequence are respectively 18-100 nucleotides in length; preferably, 30-35 nucleotides in length. In other embodiments, the tail is 2-8 nucleotides in length.

Optionally, each of the words is selected from a group consisting of CATT, CTAA, TCAT, and ACTA.

According to one exemplified embodiment, the target pathogenic microorganism belongs to Legionella spp., and the oligonucleotide has any one of the following sequences:

(SEQ ID No: 1)
GCTTGTCTTCGAGTAAGGTTAAAAACGCATTAGCGTCATCATTCTAAACT
ACGCTAATGCGTTTTTAACCTTACTCGAAGACAAGCTTTTT,
(SEQ ID No: 2)
AAAGTCTGCTTAACGCCATTGCGAGCTATGTTCGCTCATCATTCATTACT
AGCGAACATAGCTCGCAATGGCGTTAAGCAGACTTTTTTTT,
(SEQ ID No: 3)
AAACCAGGAGTAGGATTCTCTTTTAGAAAAGAATTTCATTCATACTACTA
AAATTCTTTTCTAAAAGAGAATCCTACTCCTGGTTTTTTTT,
(SEQ ID No: 4)
CGCGTGCAGTCTTATCGAATTGTTCTTCCCGGGTATCATCTAACTAACTA
ATACCCGGGAAGAACAATTCGATAAGACTGCACGCGTTTTT,
(SEQ ID No: 5)
CTCCGGATCCACCGTTTCAGGGGCTTTATTTTCTATCATCTAACATTACT
ATAGAAAATAAAGCCCCTGAAACGGTGGATCCGGAGTTTTT,
(SEQ ID No: 6)
GCTTAAGTCATAGCGTTTCCATTCTTTATTAAAGATCATCTAACATTCTA
ATCTTTAATAAAGAATGGAAACGCTATGACTTAAGCTTTTT,
(SEQ ID No: 7)
TCAATTCTGGAAATGGTGTTAAACCCGGAAAATCGTCATACTAACTACTA
ACGATTTTCCGGGTTTAACACCATTTCCAGAATTGATTTTT,
and
(SEQ ID No: 8)
ACACTGATGTTCATTTGTTAGTCTCTTTTTACAATACTACTAATCATCAT
TATTGTAAAAAGAGACTAACAAATGAACATCAGTGTTTTTT.

In another aspect, the present disclosure is directed to a kit for detecting a target pathogenic microorganism in a sample. The kit comprises a hairpin probe according to the above mentioned aspect/embodiments of the present disclosure, an internal control conjugate for quantitatively detecting the target pathogenic microorganism, and a reporter conjugate for identifying the target pathogenic microorganism.

According to one embodiment of the present disclosure, the hairpin probe is any of the above-mentioned probes. The internal control conjugate comprises a first quantum dot and an internal control probe conjugated to the first quantum dot. The first quantum dot is operable to produce a first luminescence having a first peak emission wavelength. The internal control probe has an internal control probe sequence complementary to the internal control sequence of the hairpin probe. The reporter conjugate comprises a second quantum dot and a reporter probe conjugated to the second quantum dot. The second quantum dot is operable to produce a second luminescence having a second peak emission wavelength different from the first emission wavelength. The reporter probe has a reporter probe sequence complementary to the anti-Taq sequence.

According to various optional embodiments of the present invention, the first and second quantum dots respectively comprise a core selected from the group consisting of IIB-VIB semiconductors, IIIB-VB semiconductors, and IVB-IVB semiconductors. One example of IIB-VIB semiconductors is cadmium selenide (CdSe). Still optionally, the first and second quantum dots respectively further comprise a shell selected from ZnS and CdS.

In certain embodiments, the kit may further comprise positive and/or negative control probes for confirming the validity of the detection. In one optional embodiment, the kit further comprises a positive control probe which comprises a control microbead and the internal control sequence having its 3′-end directly coupled to the control microbead. Alternatively or additionally, the kit may optionally comprise another positive control probe that comprises a control microbead and the anti-Taq sequence having its 3′-end directly coupled to the control microbead. Still optionally, the kit further comprises a negative control probe which comprises, from 5′ to 3′: the Tag sequence, the internal control sequence, anti-Tag sequence, and the tail that linked to the microbeads.

Some embodiments of the present invention allow for multiplex pathogen detection in a single reaction system, and the kit for use in multiplex pathogen detection comprises at least two different hairpin probes, at least two different internal control conjugates, and at least two different reporter conjugates. The Tag sequence of each hairpin probe is hybridizable to the specific identification sequence of only one of the at least two target pathogenic microorganisms. Also, each hairpin probe has an internal control sequence uniquely assigned thereto. The internal control probe sequence of each internal control conjugate is complementary to one of the internal control sequences, and each internal control conjugate comprises the first quantum dot operable to produce the first luminescence having the first emission wavelength different from that of the remaining first quantum dot. The reporter probe sequence of each reporter conjugate is complementary to the anti-Taq sequence of one of the hairpin probes. Also, each reporter conjugate has the second quantum dot operable to produce the second luminescence having the second emission wavelength different from the first emission wavelengths and that of the remaining second quantum dot.

In yet another aspect, the present invention is directed to a method for detecting a target pathogenic microorganism in a nucleic acid-containing sample. The present method allows for a rapid, specific, and sensitive pathogen detection. In addition, the present method permits the quantitative estimation of the pathogen in the sample. The present method is also advantageous in that it may be adapted for multiplex pathogen detection.

According to one embodiment of the present invention, the method comprises the steps as follows. In a reaction system, the nucleic acid-containing sample is mixed with the hairpin probe according to the above-mentioned aspect/embodiments. Next, the reaction system is heated to a denaturing temperature of about 95-98° C. so that the sample nucleic acid contained in the nucleic acid-containing sample and the hairpin probe are denatured, and then the reaction system is cooled to a first hybridizing temperature of about 50-55° C. for about 30 minutes to allow a first hybridization between the sample nucleic acid and the hairpin probe to proceed. Afterwards, excess amounts of the internal control conjugate and the reporter conjugate according to above-mentioned aspect/embodiments are added in the reaction system, and then a second hybridization among the sample nucleic acid, the hairpin probe, internal control conjugate, and the reporter conjugate are allowed to proceed at a second hybridizing temperature for about XX minutes. Upon the completion of the second hybridization, the unhybridized internal control conjugate and reporter conjugate are removed from the reaction system. Then, the first and second quantum dots in the reaction system are excited with an exciting wavelength, and the presence of the first luminescence and the second luminescence are detected. According to principles and spirits of the present invention, the presence of the second luminescence is indicative of the presence of the target pathogenic microorganism, whereas the number of events of the first luminescence is indicative of the amount of the target pathogenic microorganism in the sample.

In one optional embodiment of the present invention, flow cytometry analysis is used to detect the presence of the first luminescence and the second luminescence.

According to certain embodiments of the present invention, the nucleic acid-containing sample is derived from an environmental sample or a biological sample. Optionally, the nucleic acid-containing sample is used without prior nucleic acid amplification.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A and 1B depict a hairpin probe, internal control conjugate, and reporter conjugate according to one embodiment of the present invention;

FIG. 2 is flowchart illustrating a sequence of steps of a method for detecting a pathogenic microorganism according to one embodiment of the present invention;

FIG. 3A to FIG. 3E and FIG. 4A to FIG. 4E respectively illustrate the storage stability of internal control and reporter conjugates according to one example of the present invention;

FIG. 5 illustrates the standard curve of the detection kit/method according to one example of the present invention; and

FIG. 6 illustrates the specificity of the detection kit/method according to one example of the present invention.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The terms “pathogenic microorganism” and “pathogen” are used interchangeably herein to refer to any potentially harmful microorganism capable of infecting a host, or capable of eliciting a biological effect in the host when such host is exposed to said pathogenic microorganism. Pathogenic microorganisms according to the present invention include bacteria, viruses, fungi, and protozoa.

The term “sample” refers to any sample suspected of containing the target pathogenic microorganism. A sample may be an environmental sample or a biological sample. The term “environmental sample” refers to a sample collected from natural environments or man-made environments (e.g., industrial effluents, cooling water, etc). Exemplary environmental samples may be obtained or derived from soil, water, sewage, sludge, mud, air, plant and other vegetative matter, oil, liquid mineral deposits, and solid mineral deposits. The term “biological sample” means a sample that includes or is formed of a cell, tissue, or component parts (e.g., body fluids) isolated from an animal or plant. Preferably, the animal may be a human. As used herein, biological samples include clinical samples derived from subjects in need of medical treatment. Exemplary biological samples include, but are not limited to, blood, saliva, sputum, urine, feces, skin cells, hair follicles, semen, vaginal fluid, bone fragments, bone marrow, brain matter, cerebro-spinal fluid, amniotic fluid, and tissue biopsy. It should be noted that these examples are not to be construed as limiting the sample types applicable to the subject matter described herein.

The term “nucleic acid” refers to any chain of two or more nucleotides, nucleosides, or nucleobases (e.g., deoxyribonucleotides or ribonucleotides) covalently bonded together. Nucleic acids include, but are not limited to, viral genomes, or portions thereof, either DNA or RNA, bacterial genomes, or portions thereof, fungal, plant or animal genomes, or portions thereof, messenger RNA (mRNA), ribosomal RNA (rRNA), transfer RNA (tRNA), plasmid DNA, mitochondrial DNA, or synthetic DNA or RNA. A nucleic acid may be provided in a linear (e.g., mRNA), circular (e.g., plasmid), or branched form, as well as a double-stranded or single-stranded form. The term “nucleotide” is a subunit of a nucleic acid and consists of a heterocyclic base, a sugar, and one or more phosphate groups. In the most common nucleotides, the base is a derivative of purine or pyrimidine, and the sugar is the pentose deoxyribose or ribose. The purines include adenine (A), and guanine (G); the pyrimidines include cytosine (C), thymine (T), and uracil (U).

As used herein, a “sequence” of a nucleic acid refers to the ordering of nucleotides which make up a nucleic acid. Throughout this application, nucleic acids are designated as having a 5′-end and a 3′-end. Unless specified otherwise, the left-hand end of a single-stranded nucleic acid is the 5′-end; and the right-hand end of single-stranded nucleic acid is the 3′-end.

The term “hairpin” refers to any secondary structure present in a single-stranded nucleotide sequence. Specifically, the secondary structure is formed by intramolecular base pairing in the single-stranded nucleotide sequence to generate an antiparallel duplex structure (the stem) with an unpaired loop. This is the closed conformation. When the molecule is placed under a condition that prohibits the formation of the stem-loop structure, the molecule is in the open conformation.

The terms “hybridization” or “annealing” are used interchangeably to refer to the formation of complexes between nucleotide sequences which are sufficiently complementary to form complexes (or hybrids) via Watson-Crick base pairing or non-canonical base pairing. It will be understood by persons having ordinary skills in the art that the hybridization between two sequences does not require complete complementarity depending upon the stringency of the hybridization conditions. Further, the hybridization may take place between two DNA strands, two RNA strands, or one DNA and one RNA strand. The hybridization occurs under a variety appropriate conditions (e.g. temperature, pH, salt concentration, etc.) that are well known in the art of molecular biology. The term “hybrid” is used to define the complex formed between two single stranded nucleic acid sequences by said hybridization.

The term “specific identification sequence” herein refers to unique sequences of nucleotides that can be used specifically to phylogenetically define a pathogenic microorganism or a group of pathogenic microorganisms. These sequences are used to distinguish the origin of the sequence from a pathogenic microorganism at the kingdom, domain, phylum, class, order, genus, family, species and even an isolate at the phylogenic level of classification.

As used herein, the term “probe” refers to a single-stranded synthetic oligonucleotide which is designed to specifically hybridize to a target nucleic acid of complementary sequence. As used herein, a probe may include natural (i.e., A, G, C, or T) or modified bases (7-deazaguanosine, inosine, etc.) or sugar moiety. It will be understood by one of skill in the art that probes may bind specific identification sequences lacking complete complementarity with the probe sequence depending upon the stringency of the hybridization conditions. By assaying for the presence or absence of the probe, one can detect the presence or absence of the target sequence, and hence the presence or absence of a target pathogenic microorganism having the target sequence.

“Percentage (%) sequence identity” with respect to any nucleotide sequence identified herein is defined as the percentage of nucleotide residues in a candidate sequence that are identical with the nucleotide residues in the specific nucleotide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two nucleotide sequences was carried out by computer program Blastn (nucleotide-nucleotide BLAST) provided online by Nation Center for Biotechnology Information (NCBI). The percentage amino acid sequence identity of a given nucleotide sequence A to a given nucleotide sequence B (which can alternatively be phrased as a given nucleotide sequence A that has a certain % nucleotide sequence identity to a given nucleotide sequence B) is calculated by the formula as follows:

$$\frac{X}{Y} \times 100 \%$$

where X is the number of nucleotide residues scored as identical matches by the sequence alignment program BLAST in that program's alignment of A and B, and where Y is the total number of nucleotide residues in A or B, whichever is shorter.

In order to provide a rapid, specific, and sensitive pathogen detection method, a hairpin probe, an internal control conjugate, and a reporter conjugate are designed. Detailed information regarding this hairpin probe, the conjugates and the method is provided hereinbelow.

It should be noted that the following description is directed to the detection of Legionella species. Legionella is a pathogenic Gram negative bacterium with at least 50 species and 70 serogroups identified including species that cause legionellosis (Legionnaires' disease) and Pontiac fever. Legionella infections are caused by the inhalation of aerosols generated from water sources contaminated with Legionella bacteria. Legionella species are ubiquitous in many water systems including cooling towers used in industrial cooling water systems or central air conditioning systems, domestic water systems, spas, swimming pools, and fountains. It is established that L. pneumophila accounts for most clinical cases of Legionnaires' disease, while L. anisa mainly accounts for hospital acquired Legionnaires' disease and most Pontiac fever cases. For patients with suspected Legionnaires' disease, specific antibiotic treatment is necessary and should be administered promptly while diagnostic tests are being processed; and once L. pneumophila has been identified to be the causative pathogen, antimicrobial therapy directing at the pathogen shall be prescribed, and hospitalization is often required. By contrast, Pontiac fever requires no specific treatment and complete recovery usually occur within 1 week. Accordingly, the discrimination between L. pneumophila and other Legionella species is of importance for clinical treatment and disease control. Yet, conventional culture method takes about 10 days to confirm the presence of a Legionella species in the sample, and since an environmental sample usually contains more than one Legionella species, it is infeasible to distinguish L. pneumophila from other Legionella species by conventional culture method. Therefore, several Legionella species were used in the present invention to develop a rapid, specific, and sensitive pathogen detection method and kit. However, as could be appreciated by persons having ordinary skills in the art, the following description is provided for the purpose of discussion and as examples, and thus, the claims that follow should not be limited in any way by these Legionella species.

Hairpin Probe

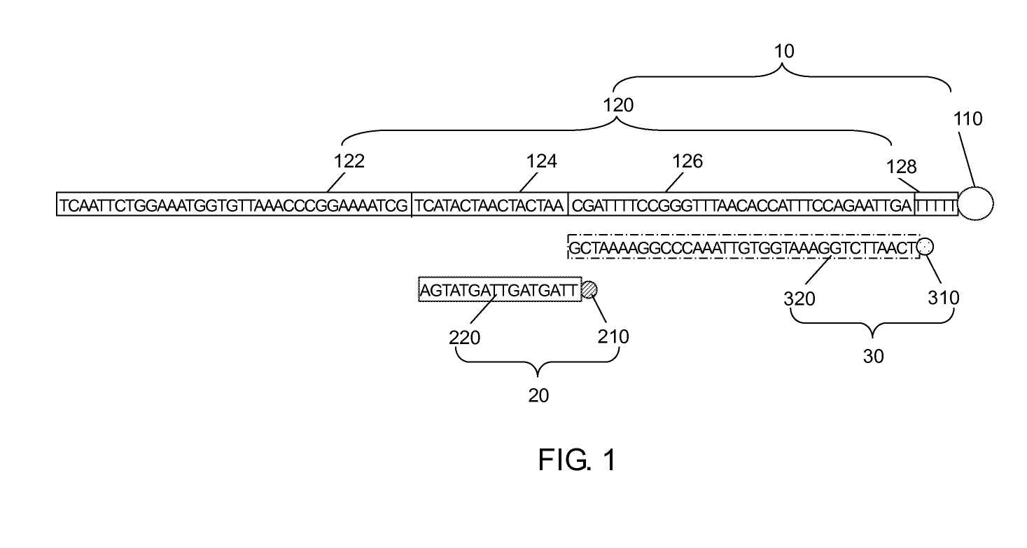

The hairpin probe design scheme is now discussed with reference to FIGS. 1A and 1B to facilitate understanding of the present disclosure. FIGS. 1A and 1B respectively depict a hairpin probe (10) in its open conformation and closed conformation. In the example illustrated in FIG. 1A, the hairpin probe (10) is designed to detect the presence of L. pneumophila in a sample. The present hairpin probe (10) has a microbead (110) and an oligonucleotide (120) having its 3′-end coupled to the microbead (110). As illustrated in FIG. 1A, the oligonucleotide (120) has a sequence of SEQ ID No: 7 (TCAATTCTGGAAATGGTGTTAAACCCGGAAAATCGTCAT ACTAACTACTAACGATTTTCCGGGTTTAACACCATTTCCAGAATTGATTTTT) and comprises, from 5′ to 3′, a Tag sequence (122), an internal control sequence (124), an anti-Tag sequence (126), and a tail (128).

According to various embodiments of the present disclosure, the microbead (110) comprises a material selected from the group consisting of polystyrene, crosslinked poly(styrene/divinylbenzene), poly(methyl methacrylate), and melamine. In the working examples provided hereinabove, the microbead is polystyrene microbead. Generally, the microbead (110) has a diameter of about 1-50 μm; preferably, about 5-8.5 μm.

The oligonucleotide (120) may be passively adsorbed on the surface of the microbead (110). Optionally, the surface of the microbead (110) may be modified to provide covalent coupling of the oligonucleotide (120). For example, the microbead (110) may be carboxylate-modified or amine-modified.

According to the principles and spirits of the present invention, the Taq sequence (122) is designed based on a unique sequence (referred to as “specific identification sequence” herein) of a target pathogenic microorganism so that the Taq sequence (122) would specifically hybridize to said specific identification sequence under suitable conditions. The target pathogenic microorganism may belong to a specific species or a variant of the species; alternatively, it may belong to a group of species or variants of a species. Therefore, the presence of the hybrid of the present hairpin probe (10) with a specific identification sequence of a target pathogenic microorganism is indicative of the presence of the target pathogenic microorganism in a sample.

In the example illustrated in FIGS. 1A and 1B, the target pathogenic microorganism is L. pneumophila, and the specific identification sequence (40) locates in the MIP gene thereof. In this case, the specific identification sequence (40) is the unique 35 nucleotides of the MIP gene from each Legionella sp. As could be appreciated, the Taq sequence (122) in the present example has 100% of sequence identity to the specific identification sequence (40); however, the present invention is not limited thereto. Rather, the Taq sequence (122) may have any suitable percentage of sequence identity with respect to the specific identification sequence (40), as long as the desired hybridization efficiency could be achieved. According to various embodiments of the present invention, the Tag sequence (122) is about 18-100 nucleotides in length; preferably about 30-50 nucleotides in length.

The internal control sequence (124) comprises at least four “words,” in which each word consists of 4 nucleotides with a 75% AT-content. The internal control sequence (124) is designed in such a way that it is unlikely to occur naturally in the genome of most pathogenic microorganisms. Therefore, it is feasible to quantify a specific pathogenic microorganism present in the sample, by quantifying the internal control sequence (124) of the hybrid formed between the hairpin probe (10) and the specific identification sequence of the target pathogenic microorganism. This is achieved by using a corresponding internal control conjugate described below. Under suitable conditions, internal control sequence (124) is operable to form the loop of the hairpin probe (10), as depicted in FIG. 1B. It is important that the internal control sequence (124) does not contain a palindromic sequence such that the internal control sequence (124) would not form a stem structure.

Optionally, each of the words is selected from a group consisting of CATT, CTAA, TCAT, and ACTA. Additional examples of the words include, but are not limited to, GATT, GTAA, TGAT, AGTA, CATA, CTAT, TCTT, and TCTA. A single “word” may appear more than once in an internal control sequence; for example, the internal control sequence (124) of SEQ ID No: 7 is “TCAT ACTA ACTA CTAA,” in which the word “ACTA” occurs twice.

The anti-Tag sequence (126) is a reverse complement of the Tag sequence (122) so that the anti-Tag sequence (126) and Tag sequence (124) are operable to form the stem of the hairpin probe, as illustrated in FIG. 1B. Since the genomes of most pathogenic microorganisms are double-stranded, the anti-Tag sequence (126) is also operable to form a hybrid with the specific identification sequence of the target pathogen under suitable conditions. In this case, the presence of the anti-Tag sequence (126) may advantageously improve the sensitivity of the pathogen detection. According to principles and spirits of the present invention, the anti-Tag sequence (126) is also operable to form a hybrid with a reporter conjugate specifically design for this purpose. This hybrid between the anti-Tag sequence (126) and the reporter conjugate serves as a confirmation means to reduce the false positive results of the present pathogen detection. Similar to the Tag sequence (122), the anti-Tag sequence (126) is about 18-100 nucleotides in length; preferably about 30-50 nucleotides in length.

The tail (128) comprises at least two consecutive thymidine residues. According to various embodiments of the present invention, the tail (128) is about 2-8 nucleotides in length. The exemplary hairpin probe (10) illustrated FIG. 1A has 5 consecutive thymidine residues as the tail (128).

In certain embodiments of the present invention, several hairpin probes have been designed to detect other Legionella species. These hairpin probes (including the hairpin probe illustrated in FIGS. 1A and 1B) as well as the target pathogenic microorganisms are summarized in Table 1.

TABLE 1

| L spp. | | |
|---|---|---|
| (SEQ ID No.) | Sequence | |
| L. anisa | Tag | GCTTGTCTTCGAGTAAGGTTAAAAACGCATTAGCG |
| (1) | Internal Control | TCATCATTCTAAACTA |
| | Anti-Tag | CGCTAATGCGTTTTTAACCTTACTCGAAGACAAGC |
| | Tail | TTTTT |
| L. | Tag | AAAGTCTGCTTAACGCCATTGCGAGCTATGTTCGC |
| birminhamensis | Internal Control | TCATCATTCATTACTA |
| (2) | Anti-Tag | GCGAACATAGCTCGCAATGGCGTTAAGCAGACTTT |
| | Tail | TTTTT |
| L. gormanii | Tag | AAACCAGGAGTAGGATTCTCTTTTAGAAAAGAATT |
| (3) | Internal Control | TCATTCATACTACTAA |
| | Anti-Tag | AATTCTTTTCTAAAAGAGAATCCTACTCCTGGTTT |
| | Tail | TTTTT |
| L. lansingensis | Tag | CGCGTGCAGTCTTATCGAATTGTTCTTCCCGGGTA |
| (4) | Internal Control | TCATCTAACTAACTAA |
| | Anti-Tag | TACCCGGGAAGAACAATTCGATAAGACTGCACGCG |
| | Tail | TTTTT |
| L. longbeachae | Tag | CTCCGGATCCACCGTTTCAGGGGCTTTATTTTCTA |
| (5) | Internal Control | TCATCTAACATTACTA |
| | Anti-Tag | TAGAAAATAAAGCCCCTGAAACGGTGGATCCGGAG |
| | Tail | TTTTT |
| L. oakridgensis | Tag | GCTTAAGTCATAGCGTTTCCATTCTTTATTAAAGA |
| (6) | Internal Control | TCATCTAACATTCTAA |
| | Anti-Tag | TCTTTAATAAAGAATGGAAACGCTATGACTTAAGC |
| | Tail | TTTTT |
| L. | Tag | TCAATTCTGGAAATGGTGTTAAACCCGGAAAATCG |
| pneumophila | Internal Control | TCATACTAACTACTAA |
| (7) | Anti-Tag | CGATTTTCCGGGTTTAACACCATTTCCAGAATTGA |
| | Tail | TTTTT |
| L. wadsworthii | Tag | ACACTGATGTTCATTTGTTAGTCTCTTTTTACAAT |
| (8) | Internal Control | ACTACTAATCATCATT |
| | Anti-Tag | ATTGTAAAAAGAGACTAACAAATGAACATCAGTGT |
| | Tail | TTTTT |

Internal Control Conjugate

According to principles and spirits of the present invention, an internal control conjugate specifically hybridizable to the internal control sequence of the above hairpin probe is provided for quantitatively detecting the target pathogenic microorganism.

According to certain embodiments of the present invention, the internal control conjugate comprises a first quantum dot and an internal control probe conjugated to the first quantum dot. The first quantum dot is operable to produce a first luminescence having a first peak emission wavelength. The internal control probe has an internal control probe sequence complementary to the internal control sequence of the hairpin probe. Optionally, the internal control probe is modified, e.g., by biotin, to increase its binding affinity to the first quantum dot.

As illustrated in FIG. 1A, the exemplary internal control conjugate (20) comprises a first quantum dot (210) and an internal control probe sequence (220) having a sequence of TTAGTAGTTAGTATGA (SEQ ID No: 15).

According to various embodiments of the present invention, the internal control probe sequence may be any of the sequences summarized in Table 2.

TABLE 2

| SEQ ID No. | Internal control probe sequence |
|---|---|
| 9 | TAGTTTAGAATGATGA |
| 10 | TAGTAATGAATGATGA |
| 11 | TTAGTAGTATGAATGA |
| 12 | TTAGTTAGTTAGATGA |
| 13 | TAGTAATGTTAGATGA |
| 14 | TTAGAATGTTAGATGA |
| 15 | TTAGTAGTTAGTATGA |
| 16 | ACTACTAATCATCATT |

In optional embodiments, the first quantum dot (210) may have a core-shell structure. The core may be formed from IIB-VIB semiconductors, IIIB-VB semiconductors, or IVB-IVB semiconductors. Some of the most widely used commercial quantum dots come with a core of cadmium selenide (CdSe), cadmium telluride (CdTe), or cadmium sulfide (CdS). Most common materials of the shell are zinc sulfide (ZnS) and CdS. For example, the shell is ZnS when the core is CdSe, CdTe, or CdS; and the shell is CdS when the core is CdSe. In the working example of the present disclosure, the quantum dots have a core of CdSe with a shell of ZnS.

Still optionally, the quantum dots may be further coated with a polymer shell that allows the quantum dots to be conjugated to biological molecules and to retain their optical properties. According to the working examples of the present disclosure, the first quantum dot (210) has a polymer shell directly coupled to streptavidin, whereas the internal control probe is modified with biotin, and the high binding affinity between biotin and streptavidin advantageously improve the conjugation efficiency between the internal control probe sequence (220) and the first quantum dot (210). However, other binding pairs in addition to streptavidin-biotin may be used in the present invention.

Generally, the first quantum dot (210) may have a size ranges from about 1 to 30 nm, and emissions from about 405-805 nm. As could be appreciated, the color of light that the first quantum dot (210) emits is strongly dependent on the particle size. For example, the peak emission wavelength of the first quantum dot (210) may be 525, 565, 585, 605, 625, 655, 705, or 800 nm.

Reporter Conjugate

According to principles and spirits of the present invention, a reporter conjugate specifically hybridizable to the anti-Tag sequence of the above hairpin probe is provided for positively confirming the presence of the target pathogenic microorganism thereby reducing the probability of false positive.

According to various embodiments of the present invention, the reporter conjugate comprises a second quantum dot and a reporter probe conjugated to the second quantum dot. The second quantum dot is operable to produce a second luminescence having a second peak emission wavelength different from the first emission wavelength. The reporter probe has a reporter probe sequence complementary to the anti-Taq sequence and thus is hybridizable to the anti-Tag sequence of the hairpin probe under suitable conditions.

The exemplary reporter conjugate (30) illustrated in FIG. 1A comprises a second quantum dot (310) and a reporter probe sequence (320) having a sequence of TCAATTCTGGAAATGGTGTTAAACCCGGAAAATCG (SEQ ID No: 23).

According to various embodiments of the present invention, the reporter probe sequence may be any of the sequences listed in Table 3.

TABLE 3

| Species | SEQ ID No. | Reporter probe sequence |
|---|---|---|
| L. anisa | 17 | GCTTGTCTTCGAGTAAGGTTAAAAACGCATTAGCG |
| L. birminghamensis | 18 | AAAGTCTGCTTAACGCCATTGCGAGCTATGTTCGC |
| L. gormanii | 19 | AAACCAGGAGTAGGATTCTCTTTTAGAAAAGAATT |
| L. lansingensis | 20 | CGCGTGCAGTCTTATCGAATTGTTCTTCCCGGGTA |
| L. longbeachae | 21 | CTCCGGATCCACCGTTTCAGGGGCTTTATTTTCTA |
| L. oakridgensis | 22 | GCTTAAGTCATAGCGTTTCCATTCTTTATTAAAGA |
| L. pneumophila | 23 | TCAATTCTGGAAATGGTGTTAAACCCGGAAAATCG |
| L. wadsworthii | 24 | ACACTGATGTTCATTTGTTAGTCTCTTTTTACAAT |

The second quantum dot (310), just like the first quantum dot (210), may optionally have a core-shell structure, and the above discussions regarding the composition, modification, structure, and peak emission wavelength of the first quantum dot (210) are equally applicable to the second quantum dot (310) here. Hence, detailed descriptions regarding the second quantum dot (310) are omitted herein for the sake of brevity and clarity.

Kit for Detecting Pathogenic Microorganism

According to certain embodiments of the present invention, the kit for detecting a target pathogenic microorganism in a sample comprises the above-described hairpin probe (e.g., hairpin probe 10), internal control conjugate (e.g., internal control conjugate 20), and reporter conjugate (e.g., reporter conjugate 30).

In certain embodiments, the kit may further comprise positive and/or negative control probes for confirming the validity of the detection.

In one optional embodiment, the kit further comprises a positive control probe which comprises a control microbead and the internal control sequence having its 3′-end directly coupled to the control microbead. Alternatively or additionally, the kit may optionally comprise another positive control probe that comprises a control microbead and the anti-Taq sequence having its 3′-end directly coupled to the control microbead. These positive control probes, unlike the hairpin probe, do not have the stem-loop structure, and are used to investigate whether the hybridization condition is normal in the absence of the sample nucleic acid.

Still optionally, the kit further comprises a negative control probe which comprises, from 5′ to 3′: the Tag sequence, the internal control sequence, anti-Tag sequence, and the tail. This negative control probe would form a stem-loop structure, and is used to monitor whether the internal control conjugate and the reporter conjugate would non-specifically bind thereto in the absence of the sample nucleic acid.

Some embodiments of the present invention allow for multiplex pathogen detection in a single reaction system. For example, the kit for use in multiplex pathogen detection (multiplex detection kit) may comprise at least two different hairpin probes, at least two different internal control conjugates, and at least two different reporter conjugates.

The Tag sequence of each hairpin probe is hybridizable to the specific identification sequence of only one of the at least two target pathogenic microorganisms. For example, the multiplex detection kit may have a hairpin probe of SEQ ID No: 1 (hairpin probe 1) and another hairpin probe of SEQ ID No: 7 (hairpin probe 7). In this case, hairpin probes 1 and 7 are designed to detect L. anisa and L. pnneumophila, respectively. Also, each hairpin probe has an internal control sequence uniquely assigned thereto. For example, the internal control sequences of the hairpin probes 1 and 7 are respectively “TCATCATTCTAAACTA” and “TCATACTAACTACTAA”.

The internal control probe sequence of each internal control conjugate is complementary to one of the internal control sequences, and each internal control conjugate comprises the first quantum dot operable to produce the first luminescence having the first emission wavelength different from that of the remaining first quantum dot. For example, the internal control conjugate designated to detect the internal sequence of hairpin probes 1 may have a first quantum dot with a peak emission wavelength of 525, whereas the internal control conjugate designated to detect the internal sequence of hairpin probes 7 may have a first quantum dot with a peak emission wavelength of 585.

The reporter probe sequence of each reporter conjugate is complementary to the anti-Tag sequence of one of the hairpin probes. Also, each reporter conjugate has the second quantum dot operable to produce the second luminescence having the second emission wavelength different from the first emission wavelengths and that of the remaining second quantum dot. For example, the reporter conjugate designated to detect the anti-Tag sequence of hairpin probes 1 may have a second quantum dot with a peak emission wavelength of 625 nm, whereas the reporter conjugate designated to detect the anti-Tag sequence of hairpin probes 7 may have a second quantum dot with a peak emission wavelength of 655 nm.

Quantum dots are known to have broad absorption spectrum which means that quantum dots capable of emitting different colors may be excited by a single excitation source. Therefore, the above mentioned first and second quantum dots are excitable by a single light source. This property together with the narrow symmetrical emission band makes quantum dots ideal for multiplex detection. With multiplex detection, several pathogenic microorganisms can be determined simultaneously with a single measurement, significantly decreasing the time and cost associated with the measurement.

According to the principles and spirits of the present invention, two quantum dots (i.e., one first and one second quantum dots) are used to detect one pathogenic microorganism, and the combination of quantum dots with 8 different peak emission wavelengths allows for the simultaneous detection and quantification of 28 (8*7/2) different pathogenic microorganisms.

The individual components of the kits can be packaged in a variety of containers, e.g., vials, tubes, microtiter well plates, bottles, and the like. Optionally, other reagents may be included in separate containers and provided with the kit; e.g., positive control samples, negative control samples, buffers, etc.

Still optionally, the kit may further comprise instructions for the use of the kit. Said instructions may include instructions as to the amount or concentration of the hairpin probe, internal control conjugate, and reporter conjugate provided. The instructions may further teach how to perform the present detection method and how to interpret the detection results. Additionally, the instructions can indicate various pathogenic microorganisms that may be detected with the kit. Instructions may be included in the kit in either printed or electronic form. Alternatively, the instructions can be provided by way of a link or internet address that provides access to instructions located on either an internet or extranet site. The internet site can be either publicly available or secure.

Method for Detecting Pathogenic Microorganism

Now that the design schemes of the present hairpin probe, internal control conjugate, and reporter conjugate and an exemplary detection kit have been discussed in detail, attention is directed to the pathogen detection method using such detection kit. It could be appreciated that the detection kit for use in the present method is designed in accordance with the scheme discussed hereinabove, and thus detailed descriptions regarding the hairpin probe, internal control conjugate, and reporter conjugate are omitted herein for the sake of brevity and clarity.

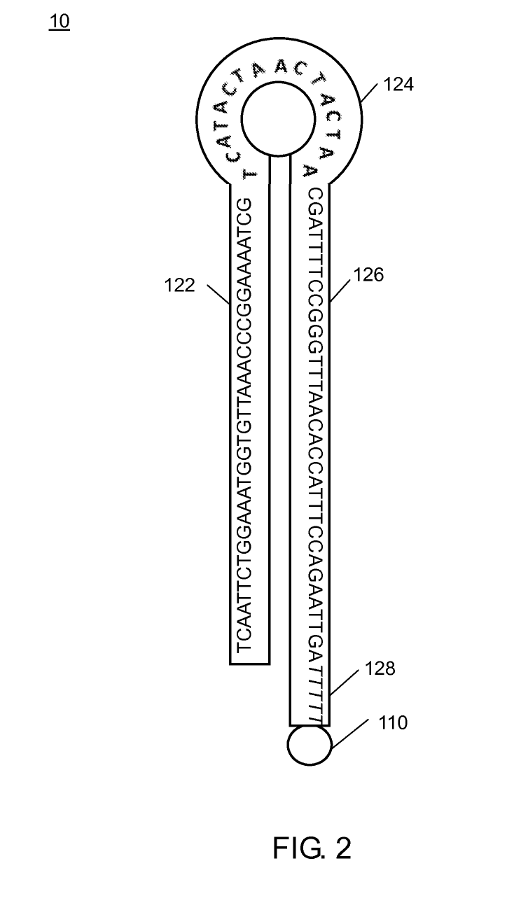

FIG. 2 is flowchart illustrating a sequence of steps of a detection method (200) according to one embodiment of the present invention, and the details of these steps are discussed further below.

First, a nucleic acid-containing sample is mixed with a present hairpin probe in a reaction system (step 205).

As could be appreciated, the present method is suitable for detecting pathogenic microorganism(s) in a sample; especially an environmental sample or a biological sample. Unlike the case of pure culture of microorganisms and cells, the environmental or biological samples often contain a variety of impurities, and hence, it is necessary to isolate the sample nucleic acid from these impurities prior to the step 205. Processing of the environmental or biological sample may involve one or more of, filtration, distillation, centrifugation, extraction, concentration, fixation, inactivation of interfering components, addition of reagents (e.g., lysis buffer), ultrasonic vibration, and the like. However, since the present method exhibit a high detecting sensitivity, it is not necessary to amplified the sample nucleic acid (e.g., by PCR-base techniques or equivalents thereof) prior to the step 205 according to certain embodiments of the present invention. Similarly, in some embodiments, no prior cell culture is required before the step 205.

According to various embodiments, the hairpin probe is added in an amount of about 10-50 pmoles, and preferably about 20-30 pmoles. For example, the hairpin probe may be present in an amount of about 10, 15, 20, 25, 30, 35, 40, 45, or 50 pmoles. In one working example, about 25 pmoles of the hairpin probe is present in the reaction system.

Next in step 210, the reaction system is heated to a denaturing temperature of about 95-98° C. so that the sample nucleic acid contained in the nucleic acid-containing sample and the hairpin probe are denatured, and then the reaction system is cooled to a first hybridizing temperature of about 50-55° C. for a first hybridizing time of about 30 minutes to allow a first hybridization between the sample nucleic acid and the hairpin probe to proceed.

The denaturing and hybridizing conditions are determined depending on factors such as the sequence and length of the hairpin probe and the sample nucleic acid. Generally, the denaturing temperature is about 95-98° C., and the denaturing time is about 1 to 10 minutes. Specifically, the denaturing temperature may be 95, 96, 97, or 98° C., and the denaturing time may be 1, 2, 3, 4, 5, 6, 7, 8, 9, or 10 minutes. For examples, the denaturing condition used in the following working examples was 95° C. for 10 minutes. The first hybridizing temperature is about 50-55° C., and specifically, about 50, 51, 52, 53, 54, or 55° C. The exemplary first hybridizing temperature used in the working examples was about 50° C.

If the sample contains nucleic acid derived from the target pathogenic microorganism, the hairpin probes may hybridize with the specific identification sequence of the target pathogenic microorganism under the first hybridization condition. This hairpin probes would no longer maintain the stem-loop structure

According to principles and spirits of the present invention, it is vital that the first hybridization between the sample nucleic acid and the hairpin probe is not completed. Based on the hybridization kinetics and prior experiences, complete hybridization between the sample nucleic acid and the hairpin probe is achieved in about 1 hour, and therefore, the first hybridizing time is set to be about 20-40 minutes. Specifically, the first hybridizing time may be 20, 25, 30, 35, or 40 minutes. In the working examples provided hereinbelow, the first hybridizing time was about 30 minutes.

Afterwards, the method proceeds to step 215 in which excess amounts of a present internal control conjugate and a present reporter conjugate are added in the reaction system, and then a second hybridization among the sample nucleic acid, the hairpin probe, internal control conjugate, and the reporter conjugate is allowed to proceed at a second hybridizing temperature for a second hybridizing time of about 30 minutes.

Generally, the “excess amount” means that a conjugate is present at a higher molar concentration than the hairpin probe. Typically, when present in excess, the internal control conjugate or the reporter conjugate may be present at least a 10-fold molar excess; for example, at least 250 pmoles of each conjugate would be used when the hairpin probe was present at about 25 pmoles or less.

As discussed earlier, the first hybridization does not allow a complete hybridization, and hence there are some unhybridized sample nucleic acids and hairpin probes in the reaction system. Under this circumstance, combine with the fact that the reporter conjugate is given in excess amount, the reporter conjugate would compete with the sample nucleic acid for hybridization with the anti-Tag sequence.

The hybridization of the hairpin probe with the sample nucleic acid and/or the reporter conjugate would keep the hairpin probe in its open conformation. As a result, the internal control probe would have the chance to hybridize with the internal control sequence of the open hairpin probe.

According to principles and spirits of the present invention, this second hybridization shall allow a complete hybridization of the hairpin probe with the sample nucleic acid, internal control conjugate, and/or the reporter conjugate. Based on the hybridization kinetics and prior experiences, 30 to 120 minutes ensure a complete reaction. Therefore, the second hybridizing time is about 30-120 minutes; e.g., 30, 35, 40, 45, 50, 55, 60, 65, 70, 75, 80, 85, 90, 95, 100, 105, 110, 115, or 120 minutes. In the working examples provided hereinbelow, the first hybridizing time was about 60-120 minutes.

The second hybridizing temperature is about 50-55° C., and specifically, about 50, 51, 52, 53, 54, or 55° C. Moreover, the second hybridizing temperature may be the same as or different from the first hybridizing temperature. The exemplary second hybridizing temperature used in the working examples was about 50° C.

Next, in step 220, the unhybridized internal control conjugate and reporter conjugate are removed from the reaction system upon the completion of the second hybridization. For example, the reaction system may be centrifuge to allow the sedimentation of a microbead-containing mixture. Then, the microbead-containing mixture may be washed with PBS buffer to remove the unhybridized internal control conjugate and reporter conjugate.

Then, the first and second quantum dots in the reaction system are excited with an exciting wavelength of about 400-650 nm (preferably, 400-500 nm), and the presence of the first luminescence and the second luminescence are detected. For example, a flow cytometer with a suitable optical filter may be used in step 220 to detect the presence of the first luminescence and the second luminescence.

As could be appreciated, since the reporter probe sequence of the reporter conjugate is specific to the anti-Tag sequence of the hairpin probe, it is feasible to positively confirm the presence of the specific identification gene in the sample by detecting the presence of the hybrid between the reporter conjugate and the hairpin probe; e.g., by detecting the second luminescence emitted from the second quantum dot of the reporter conjugate. In addition, since the internal control conjugate is used in excess amount in relative to the hairpin probe, theoretically, each open hairpin probe would has an internal control conjugate attached to the internal control sequence thereof. Accordingly, it is feasible to quantify the amount of the target pathogenic microorganism present in the sample by counting the events of the first luminescence emitted from the first quantum dot of the internal control conjugate.

In view of the foregoing, the present method 200 exhibits the potential to detect and identify a pathogenic microorganism in environmental or biological samples within a few hours without time-consuming cultivation and/or amplification required by prior techniques. Therefore, the present method is a rapid and highly valuable method for the specific identification of a pathogenic microorganism. Moreover, the present method 200 is useful for quantitative estimation of the pathogen in the sample.

As could be appreciated, the above method 200 is directed to the detection of a single pathogenic microorganism; however, the present invention is not limited thereto. In other embodiments, the present method is suitable for use in multiplex pathogen detection by using the above-mentioned multiplex detection kit. The steps for multiplex detection are substantially similar to those of the detection method 200, and thus are omitted herein for the sake of brevity and clarity.

The possibility of the rapid and multiplex detection of more than one pathogenic microorganism in a sample is advantageous in various fields including environmental quality management, disease control, and clinical management.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example I

### Preparation of Hairpin Probe

Oligonucleotide sequences (SEQ ID Nos: 1-8) were purchased from Invitrogen (Taiwan). Polystyrene microbeads (Micro particles based on polystyrene, carboxylate-modified, average diameter: 8.5 μm) were obtained from Sigma-Aldrich (St. Louis, Mo.).

The oligonucleotide was coupled to the microbead surface using the protocol as follows. 5,000,000 microbeads were suspended in 2-(N-morpholino)ethanesulfonic acid (MES buffer, pH 4.5, 350 μL), and 50 mg of N-(3-Dimethylaminopropyl)-N′-ethylcarbodiimide hydrochloride (EDC, Sigma-Aldrich) in MES buffer (pH 4.5, 150 μl) was added. A constant amount of the oligonucleotide stock solution (82 pmol) was added to varying concentrations of said microbead-containing suspension such that the microbeads to oligonucleotides molar ratio was about 1:1, 1:10, 1:100, or 1:1000. The reaction mixture was then left to react on a mixer overnight. The reaction mixture was centrifuged for 15 minutes at 2400 g and washed with MEST solution (MES buffer, 0.1% Tween, pH 4.5, 300 μL) to remove nonspecifically bound oligonucleotides, and the microbeads were collected and stocked in a stabilizing buffer (0.1% PEG4000) at 4° C. for characterization and further use.

Total coupling efficiency was determined by measuring the optical density OD₂₆₀ and OD_{260/280} ratio in the supernatant using a spectrophotometer. The coupling efficiencies of each hairpin probe at various microbeads/oligonucleotides ratios were summarized in Table 4, in which the coupling ratio refers to the numbers of oligonucleotide strands per microbead. The hairpin probes 1-8, indicated below, respectively refer to hairpin probes having an oligonucleotide of SEQ ID Nos: 1-8.

TABLE 4

| Hair- | | | | | |
|---|---|---|---|---|---|
| pin | | Microbeads/oligonucleotides molar ratio | | | |
| Probe | | 1:1 | 1:10 | 1:100 | 1:1000 |
| 1 | OD₂₆₀* | 0.322 | 0.103 | 0.128 | 0.18 |
| | OD_{260/280} | 2.01 | 3.81 | 2.84 | 3.6 |
| | Coupling ratio** | 7.51 × 10⁹ | 2.40 × 10⁹ | 3.00 × 10⁹ | 4.18 × 10⁹ |
| 2 | OD₂₆₀* | 1.106 | 0.144 | 0.32 | 0.096 |
| | OD_{260/280} | 1.86 | 2.82 | 2.29 | 7.38 |
| | Coupling ratio** | 7.80 × 10⁸ | 3.30 × 10⁹ | 7.44 × 10⁹ | 2.23 × 10⁹ |
| 3 | OD₂₆₀* | 1.181 | 0.259 | 0.182 | 0.172 |
| | OD_{260/280} | 1.9 | 2.25 | 2.46 | 3.58 |
| | Coupling ratio** | 2.74 × 10⁹ | 6.02 × 10⁹ | 4.25 × 10⁹ | 4.00 × 10⁹ |
| 4 | OD₂₆₀* | 0.208 | 0.111 | 0.102 | 0.089 |
| | OD_{260/280} | 2.04 | 2.52 | 3.19 | 4.05 |
| | Coupling ratio** | 4.85 × 10⁹ | 2.59 × 10⁹ | 2.38 × 10⁹ | 2.07 × 10⁹ |
| 5 | OD₂₆₀* | 0.208 | 0.115 | 0.128 | 0.209 |
| | OD_{260/280} | 1.93 | 2.61 | 32 | 2.38 |
| | Coupling ratio** | 4.85 × 10⁹ | 2.68 × 10⁹ | 3.00 × 10⁹ | 4.89 × 10⁹ |
| 6 | OD₂₆₀* | 0.39 | 0.118 | 0.066 | 0.229 |
| | OD_{260/280} | 2.17 | 2.74 | 4.4 | 6.54 |
| | Coupling ratio** | 9.11 × 10⁹ | 2.76 × 10⁹ | 1.54 × 10⁹ | 5.35 × 10⁹ |
| 7 | OD₂₆₀* | 0.303 | 0.04 | 0.198 | 0.154 |
| | OD_{260/280} | 2.09 | 1.43 | 2.44 | 2.85 |
| | Coupling ratio** | 7.05 × 10⁹ | 9.32 × 10⁹ | 4.60 × 10⁹ | 3.50 × 10⁹ |
| 8 | OD₂₆₀* | 0.269 | 0.162 | 0.51 | 0.214 |
| | OD_{260/280} | 2.2 | 3.77 | 3.00 | 4.98 |
| | Coupling ratio** | 6.27 × 10⁹ | 3.75 × 10⁹ | 1.19 × 10⁹ | 5.00 × 10⁹ |

*Data represent mean from three independent replicates.
**Coupling ratio = (Value of OD₂₆₀ × 33 ug/ml)/(330 × No. of oligonucleotides) × 6 × 10²³/No. of microbeads.

### Example II

### Preparation and Storage Stability of Internal Control Conjugate and Reporter Conjugate

Biotinylated internal control probe sequences (SEQ ID Nos: 9-16), biotinylated reporter probe sequence (SEQ ID Nos: 17-24), and streptavidin-modified quantum dots (Q10131MP, peak emission: 565 nm; and Q10101MP peak emission: 605 nm) were purchased from Invitrogen (Taiwan).

Biotinylated internal control probe sequences were mixed with Q10101MP quantum dots with a molar ratio of 1:1, and biotinylated reporter probe sequences were mixed with Q10131MP quantum dots with a molar ratio of 1:1. The mixture was allowed to react at 37° C. for 1 hour followed by purification through Sephadex G50 column to remove unconjugated quantum dots. Optionally, a second purification was conducted by passing the mixture obtained from the Sephadex G50 column through Sephadex G75 column to remove unconjugated probes, thereby further improving the sensitivity of the present method and kit.

The conjugated quantum dots were collected and stocked for characterization and further use. Conjugating efficiency was determined by measuring the optical density OD₂₆₀ and OD₅₆₅ or OD₆₀₅ in the supernatant using a spectrophotometer. The conjugating efficiencies of conjugates were summarized in Table 5. The internal control conjugates 1-8 respectively have an internal control probe sequence of SEQ ID Nos: 9-16, whereas the reporter conjugate 1-8 respectively have a reporter probe sequence of SEQ ID Nos: 17-24.

TABLE 5

| Internal | | | | | |
|---|---|---|---|---|---|
| control | | | DNA con | | Conjugating |
| conjugate | | OD₂₆₀ | (ng/μl) | OD₆₀₅ | efficiency* (%) |
| 1 | Blank | 2.08 | 68.64 | 0 | — |
| | G50 | 2.08 | 68.64 | 10 | 10 |
| | G50 + G75 | 1.4 | 46.2 | 19 | 57.4 |
| 2 | Blank | 2.3 | 75.9 | 0 | — |
| | G50 | 2.02 | 66.66 | 8 | 8 |
| | G50 + G75 | 1.48 | 48.8 | 28 | 84.7 |
| 3 | Blank | 2.13 | 70.29 | 0 | — |
| | G50 | 1.60 | 52.8 | 18 | 23 |
| | G50 + G75 | 3.7 | 122.1 | 28 | 84 |
| 4 | Blank | 2.02 | 66.66 | 0 | — |
| | G50 | 2.03 | 66.99 | 11 | 11 |
| | G50 + G75 | 3.7 | 122.1 | 31 | 37.2 |
| 5 | Blank | 2.1 | 69.3 | 0 | — |
| | G50 | 2.07 | 68.31 | 14 | 14 |
| | G50 + G75 | 1.5 | 49.5 | 20 | 60 |
| 6 | Blank | 2.06 | 67.98 | 0 | — |
| | G50 | 2.53 | 83.49 | 16 | 13 |
| | G50 + G75 | 0.62 | 20.7 | 10 | 71.2 |
| 7 | Blank | 2.13 | 70.29 | 0 | — |
| | G50 | 1.44 | 47.52 | 13 | 18 |
| | G50 + G75 | 3.7 | 122 | 40 | 48 |
| 8 | Blank | 2.07 | 68.31 | 0 | — |
| | G50 | 1.51 | 49.83 | 5 | 6 |
| | G50 + G75 | 4.7 | 155.1 | 51 | 48.2 |
| Reporter | | | DNA con | | Conjugating |
| conjugate | | OD₂₆₀ | (ng/μl) | OD₅₆₅ | efficiency* (%) |
| 1 | Blank | 1.82 | 60.06 | 0 | — |
| | G50 | 1.98 | 65.34 | 9 | 20 |
| | G50 + G75 | 1.45 | 47.85 | 32 | 98 |
| 2 | Blank | 1.7 | 56.1 | 0 | — |
| | G50 | 1.73 | 57.1 | 8 | 21 |
| | G50 + G75 | 4.80 | 158.4 | 50 | 47 |
| 3 | Blank | 1.99 | 65.67 | 0 | — |
| | G50 | 1.97 | 65.01 | 7 | 16 |
| | G50 + G75 | 1.64 | 54.12 | 32 | 87 |
| 4 | Blank | 1.46 | 48.18 | 0 | — |
| | G50 | 1.83 | 60.39 | 8 | 20 |
| | G50 + G75 | 1.48 | 48.84 | 32 | 96 |
| 5 | Blank | 1.47 | 48.51 | 0 | — |
| | G50 | 2.13 | 70.29 | 8 | 17 |
| | G50 + G75 | 1.36 | 44.88 | 24 | 78 |
| 6 | Blank | 1.72 | 56.76 | 0 | — |
| | G50 | 1.85 | 61.05 | 7 | 17 |
| | G50 + G75 | 3.72 | 122.76 | 39 | 46 |
| 7 | Blank | 1.81 | 59.73 | 0 | — |
| | G50 | 2.02 | 66.66 | 9 | 20 |
| | G50 + G75 | 3.69 | 121.77 | 43 | 52 |
| 8 | Blank | 1.71 | 56.43 | 0 | — |
| | G50 | 2.36 | 77.88 | 10 | 19 |
| | G50 + G75 | 4.72 | 155.76 | 40 | 37 |

*Conjugating efficiency: ((Value of OD₂₆₀ × 33/(330 × No. of primer bases)) × 6.02 × 10²³)/(Value of OD₆₀₅ or OD₅₆₅ × 7.69 × 10¹⁹).

As can be seen in Table 5, the conjugating efficiencies of the internal control conjugates or the reporter conjugates were substantially improved by the second purification process with Sephadex G75 column.

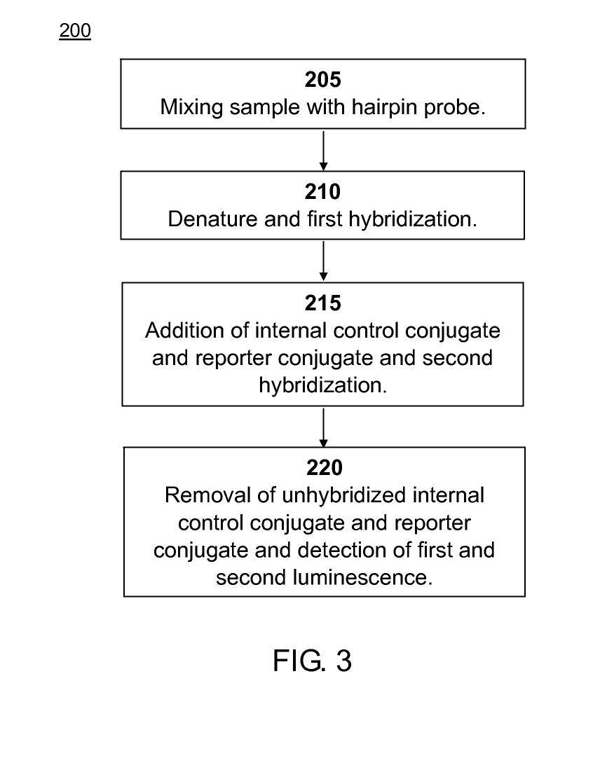

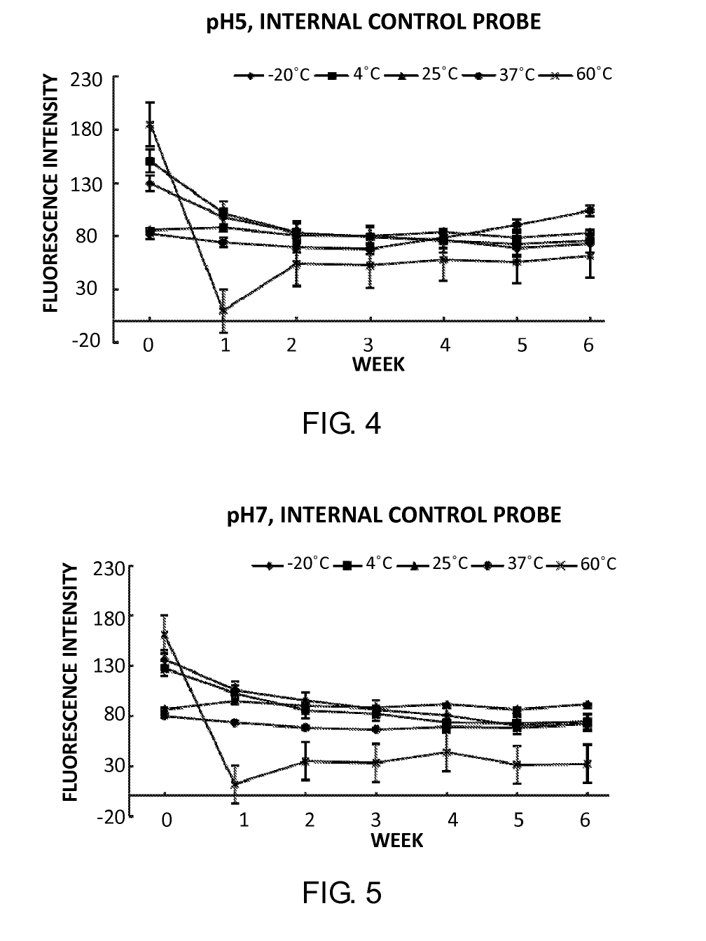

The internal control and reporter conjugates were stored under various conditions to investigate the storage stability thereof, and the results were summarized in FIG. 3A to FIG. 3E and FIG. 4A to FIG. 4E.

Specifically, FIG. 3A illustrates the relationships between fluorescence intensity and time when the internal control conjugate with quantum dot 605 nm was stored at pH 5 and various storage temperatures (including about −20, 4, 25, 37, and 60° C.). For internal control conjugate with quantum dot 605 nm stored at pH 7, 9, 11, and 13, the fluorescence-time relationships are illustrated in FIG. 3B to FIG. 3E, respectively. Data illustrated in FIG. 3A to FIG. 3 indicate that the high storage temperature of about 60° C. had deleterious effect on storage with the fluorescence intensity markedly dropped to about one-tenth of the initial intensity within a week. On the other hand, there were no significant differences observed among storage temperatures of about −20, 4, 25, and 37° C. for internal control conjugates stored at the same pH. Therefore, the storage temperature tanged between −20-37° C. did not appear to substantially affect the storage stability of internal control conjugates. Strong alkali conditions (e.g. pH11-13) also adversely affect the storage stability of internal control conjugates and significant reductions of fluorescence intensity took place in less than a week. In sum, the present internal control conjugates exhibited satisfactory storage stability when stored under suitable storage conditions (e.g., about −20-37° C. and pH 3-9), and had a storage life of at least 6 weeks.

FIG. 4A to FIG. 4E illustrate the fluorescence-time relationships of reporter conjugates with quantum dot 565 nm stored at various temperature and pH 5, 7, 9, 11, and 13, respectively. Similar to the observation of internal control probes; high storage temperature and strong alkali condition were unfavorable for the storage stability of reporter probes. High storage temperature of about 60° C. caused the fluorescence intensity of reporter conjugates to decrease to about one-third of the initial intensity in less than a week. Also, significant drops in fluorescence intensity of the reporter conjugates were also observed in strong alkali conditions (e.g. pH11-13) within a week. Together, FIG. 4A to FIG. 4E illustrate that the present reporter conjugates exhibited satisfactory storage stability when stored under suitable storage conditions (e.g., about −20-37° C. and pH 3-9), and had a storage life of at least 4 weeks.

### Example III

### Detection Limit and Linearity

The analytical sensitivity, or limit of detection (LOD), was determined using L. pneumophila cells and DNA extracted therefrom.

L. pneumophila cell culture which was obtained from American Type Culture Collection (ATCC, Manassas, Va.) was cultured on a buffered charcoal yeast extract (BCYE) medium at 35° C. in 2.5% CO₂ for 48-72 hours, and then a single colony was transferred to a fresh BCYE medium and incubated at 35° C. in 2.5% CO₂ for 48-72 hours. The colonies were harvested, adjusted to 10⁹ CFU/ml in distilled water and aliquoted at −70° C. until used. Bacterial genomic DNA was extracted from L. pneumophila cells and confirmed by ONAR® LS Legionella spp. Detection Kit, followed by ultrasonic vibration for about 10 min to give DNA fragments of about 200-250 bp.

A L. pneumophila stock (concentration [symbol] 10⁹CFU/ml) was serially diluted over 6 logs (i.e., 10³-10⁹ CFU/ml). The diluted stock (1 ml) was mixed with 1 μl hairpin probe 7 (10⁵ microbeads/μl) and heated to 95° C. for 5 min; then cooled to 50° C. for 10 min. Afterwards, internal control conjugate 7 and reporter control conjugate 7 (molar ratio: 1:1) about 100 pmole in each were added to the mixture and maintained at 50° C. for 1-2 hours. The mixture was centrifuged and then washed with PBS buffer and subjected to flow cytometry analysis (flow cytometer model; excitation: 488 nm; filter: 564-606 nm). Three independent replicates at each dilution were tested, and means from the three replicates are summarized in Table 6.

TABLE 6

| | Cell Counts (CFU/ml) | | | | | | |
|---|---|---|---|---|---|---|---|
| | 10⁹ | 10⁸ | 10⁶ | 10⁵ | 10⁴ | 10³ | 0 |
| Events for 565 nm | 9585 | 9874 | 9909 | 1217 | 450 | 54 | 47 |
| Events for 605 nm | 9877 | 9947 | 9856 | 816 | 448 | 50 | 45 |
| 565 nm/605 nm | 0.97 | 0.99 | 1.01 | 1.49 | 1.00 | 1.08 | 1.05 |

The data in Table 6 suggest that the lowest concentration resulting in a positive result was 10⁴ CFU/ml, while for a concentration lower than 10⁴ CFU/ml, the detected counts were indistinguishable from background (negative control, cell counts: 0 CFU/ml).

Fragments of genomic DNA were also serially diluted to various concentrations, and then mixed with the present hairpin probe 7, internal control conjugate 7, and reporter conjugate 7 and analyzed as described above. Three independent replicates at each dilution were tested, and means from the three replicates are summarized in Table 7.

TABLE 7

| | DNA amount (ng) | | | | | | | |
|---|---|---|---|---|---|---|---|---|
| | 1000 | 500 | 250 | 100 | 50 | 10 | 1 | 0 |
| Events for 565 nm | 9854 | 6036 | 5032 | 1410 | 609 | 130 | 20 | 10 |
| Events for 605 nm | 9947 | 5967 | 4622 | 1217 | 540 | 120 | 35 | 8 |
| 565 nm/605 nm | 0.99 | 1.01 | 1.09 | 1.16 | 1.38 | 1.08 | 0.57 | 1.25 |

As can be seen in Table 7, the lowest DNA amount resulting in a positive result was 10 ng, while for a DNA amount lower than 10 ng, the detected counts were indistinguishable from background (negative control, DNA amount: 0 ng).

FIG. 5 illustrates the standard curve plotted with data summarized in Table 5. Results demonstrated linearity (r²=0.9838) of L. pneumophila detection as a function of DNA concentration input over 3 logs.

### Example IV

### Analytical Specificity

Commercially obtained, purified genomic DNA from 8 Legionella spp. (Group A, including, L. anisa, L. birminhamensis, L. gormanii, L. lansingensis, L. longbeachae, L. oakridgensis, L. pneumophila, and L. wadsworthi; all from ATCC) and 8 other bacterial strains (Group B, including, Escherichia coli DH5α from Invitrogen; Vibrio vulnifius CG021 courtesy from Prof. Lien-I Hor (Department of Microbiology and Immunology, National Cheng Kung University, Tainan, Taiwan); and Bacillus subtilis, Proteus mirabilis, Streptococcus pneumonia, Vibrio cholera, L. cherri, and L. dumoffil, all from Bioresource Collection & Research Center (BCRC)) were tested. Each cell line was cultured and the genomic DNA was extracted, as described in Example III above.

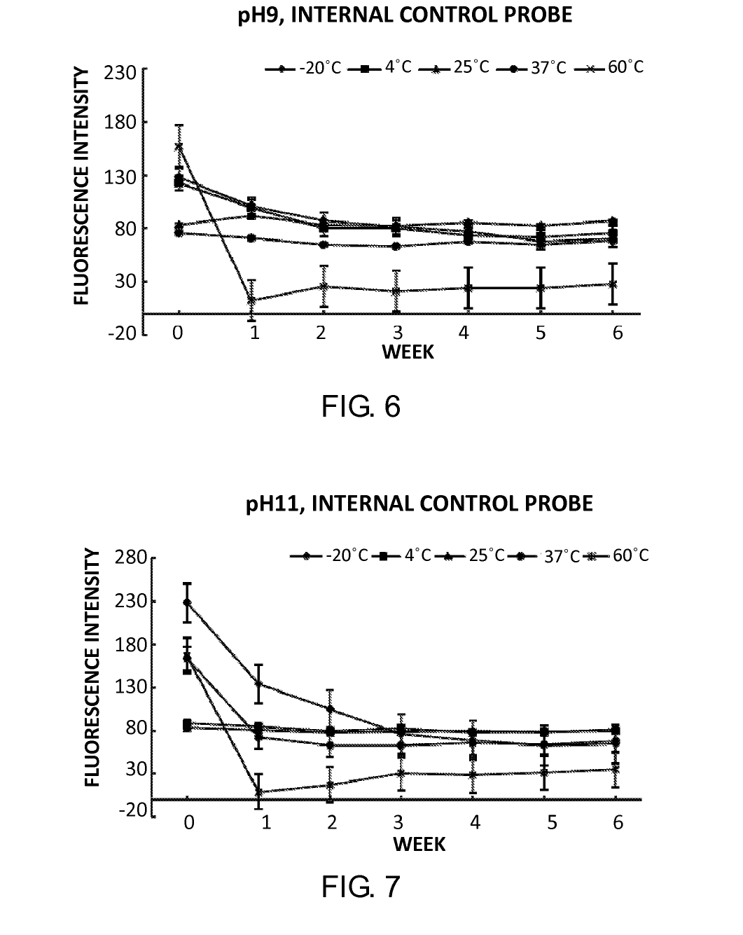

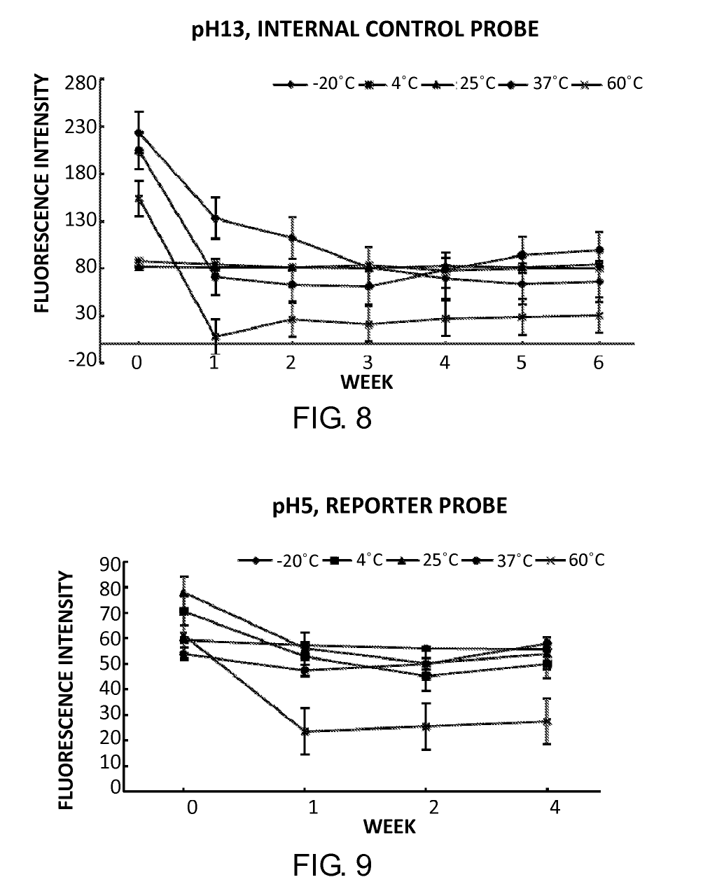

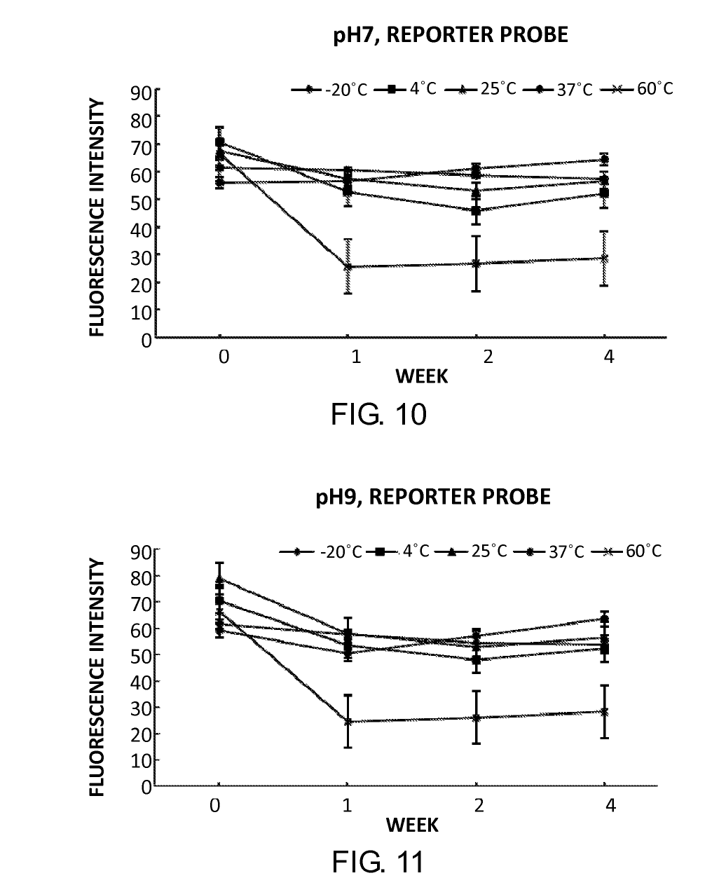

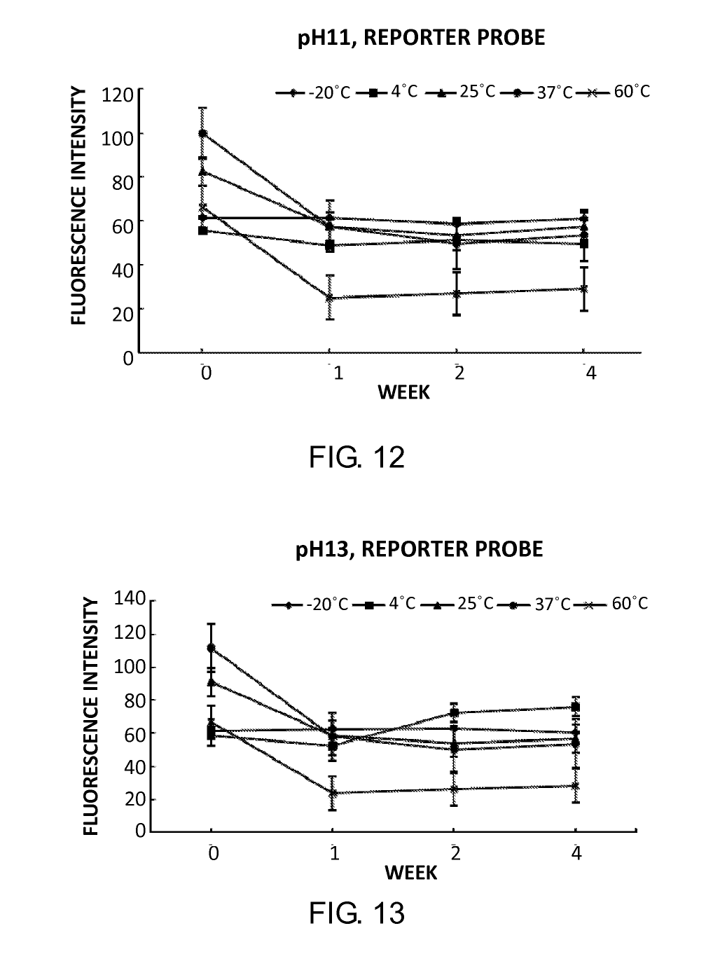

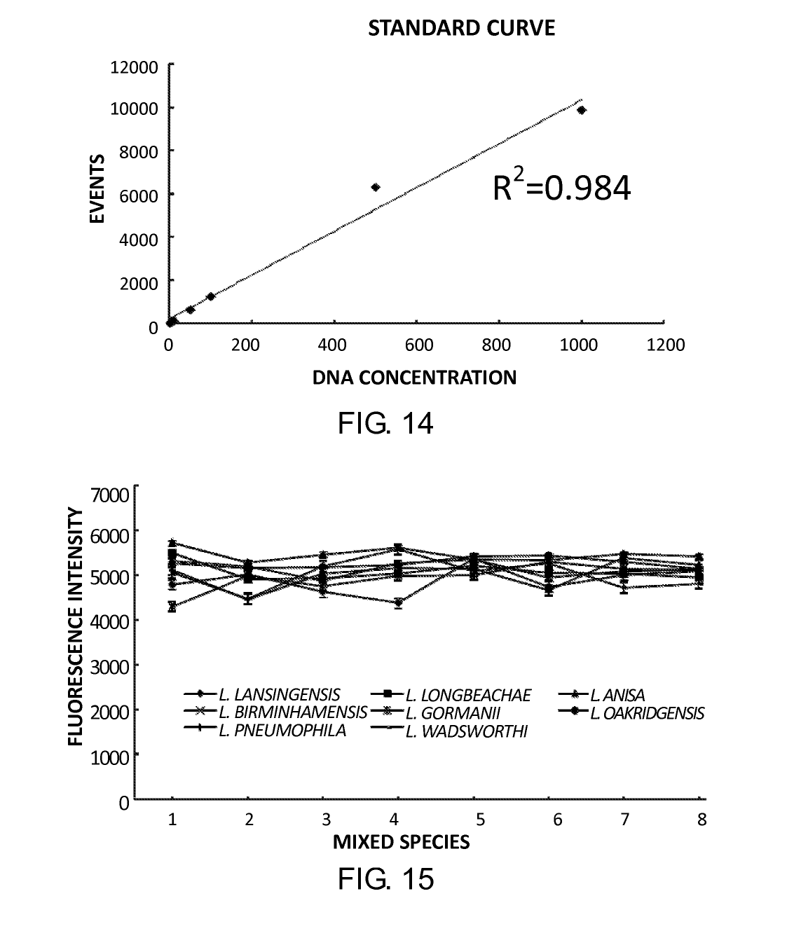

For the specificity with respect to other Legionella spp., a target Legionella strain (500 ng) was mixed 1 to 7 other Legionella strains from Group A (100 ng each). The sample was then mixed with the respective hairpin probe, internal conjugate, and reporter conjugate and analyzed as described in Example III. For example, to investigate the specificity of L. anisa among other Legionella strains, samples containing L. anisa alone (strain number: 1), L. anisa+L. birminhamensis (strain number: 2), L. anisa+L. birminhamensis+L. gormanii (strain number: 3), L. anisa+L. birminhamensis+L. gormanii+L. lansingensis (strain number: 4), L. anisa+L. birminhamensis+L. gormanii+L. lansingensis+L. longbeachae (strain number: 5), L. birminhamensis+L. gormanii+L. lansingensis+L. longbeachae+L. oakridgensis (strain number: 6), L. birminhamensis+L. gormanii+L. lansingensis+L. longbeachae+L. oakridgensis+L. pneumophila (strain number: 7), and L. birminhamensis+L. gormanii+L. lansingensis+L. longbeachae+L. oakridgensis+L. pneumophila+L. wadsworthi (strain number: 8) were prepared. Each sample was then mixed with the above-mentioned hairpin probe 1, internal control conjugate 1, and reporter conjugate 1 in accordance with steps set forth in Example III. Three independent replicates at each dilution were tested, and means from the three replicates are summarized in FIG. 6.

Results in FIG. 6 indicate that the present method/kit exhibited satisfactory specificity for detecting a target Legionella strain in a sample containing various other Legionella spp. Also, the presence of other Legionella strains in the sample would not substantially interfere with the specificity of the present method/kit to the target Legionella strain.

For the specificity with respect to other bacterial, eight bacterial strains were mixed with equal parts and then aliquoted to various concentrations and then mixed with a target Legionella strain (L. pneumophila, 500 ng). Each sample was then mixed with the above-mentioned hairpin probe 7, internal control conjugate 7, and reporter conjugate 7 in accordance with steps set forth in Example III. Three independent replicates at each dilution were tested, and means from the three replicates are summarized in Table 6.

TABLE 6

| | DNA concentration | | | | Positive | Negative |
|---|---|---|---|---|---|---|
| | 5 μg | 3 μg | 500 ng | 250 ng | Control | Control |
| Events for | 6325 | 5653 | 6154 | 5301 | 5064 | 836 |
| 565 nm | | | | | | |
| Events for | 5883 | 5925 | 6347 | 5898 | 6097 | 727 |
| 605 nm | | | | | | |

As can be seen in Table 6, the present method/kit exhibited satisfactory specificity for detecting a target Legionella strain in a sample containing a combination of gram positive and gram negative bacteria. Further, the presence of other bacterial strains in the sample would not substantially interfere with the specificity of the present method/kit to the target Legionella strain. Accordingly, the false positive probability may be reduced.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A hairpin probe for detecting a target pathogenic microorganism in a sample, wherein the target pathogenic microorganism belongs to Legionella spp. and the hairpin probe has a stern-and-loop structure and comprises a microbead having a diameter of 1-50 μm, and an oligonucleotide having its 3′-end coupled to the microbead, wherein the oligonucleotide consists essentially of, from 5′ to 3′:
a Tag sequence hybridizable to a specific identification sequence of the target pathogenic microorganism;
an internal control sequence comprising at least four words, wherein each word consists of 4 nucleotides with a 75% AT-content, wherein the internal control sequence is operable to form the loop;
an anti-Tag sequence being a reverse complement of the Tag sequence so that the anti-Tag sequence and the Tag sequence are operable to form the stem; and
a tail comprising at least two consecutive thymidine residue, and the oligonucleotide has any one of the following sequences:
(SEQ ID No: 1)
GCTTGTCTTCGAGTAAGGTTAAAAACGCATTAGCGTCATCATTCTAAACT
ACGCTAATGCGTTTTTAACCTTACTCGAAGACAAGCTTTTT,
(SEQ ID No: 2)
AAAGTCTGCTTAACGCCATTGCGAGCTATGTTCGCTCATCATTCATTACT
AGCGAACATAGCTCGCAATGGCGTTAAGCAGACTTTTTTTT,
(SEQ ID No: 3)
AAACCAGGAGTAGGATTCTCTTTTAGAAAAGAATTTCATTCATACTACTA
AAATTCTTTTCTAAAAGAGAATCCTACTCCTGGTTTTTTTT,
(SEQ ID No: 4)
CGCGTGCAGTCTTATCGAATTGTTCTTCCCGGGTATCATCTAACTAACTA
ATACCCGGGAAGAACAATTCGATAAGACTGCACGCGTTTTT,
(SEQ ID No: 5)
CTCCGGATCCACCGTTTCAGGGGCTTTATTTTCTATCATCTAACATTACT
ATAGAAAATAAAGCCCCTGAAACGGTGGATCCGGAGTTTTT,
(SEQ ID No: 6)
GCTTAAGTCATAGCGTTTCCATTCTTTATTAAAGATCATCTAACATTCTA
ATCTTTAATAAAGAATGGAAACGCTATGACTTAAGCTTTTT,
(SEQ ID No: 7)
TCAATTCTGGAAATGGTGTTAAACCCGGAAAATCGTCATACTAACTACTA
ACGATTTTCCGGGTTTAACACCATTTCCAGAATTGATTTTT,
and
(SEQ ID No: 8)
ACACTGATGTTCATTTGTTAGTCTCTTTTTACAATACTACTAATCATCAT
TATTGTAAAAAGAGACTAACAAATGAACATCAGTGTTTTTT.

2. The hairpin probe of the claim 1, wherein the microbead comprises carboxylate-modified polystyrene.

3. (canceled)

4. The hairpin probe of the claim 1, wherein the diameter is 5-8.5 μm.

5. The hairpin probe of the claim 1, wherein the Tag sequence and the anti-Tag sequence are respectively 18-100 nucleotides in length.

6. The hairpin probe of the claim 1, wherein each of the words is selected from a group consisting of CATT, CTAA, TCAT, and ACTA.

7. The hairpin probe of the claim 1, wherein the tail is 2-8 nucleotides in length.

8. (canceled)

9. A kit for detecting a target pathogenic microorganism in a sample, wherein the target pathogenic microorganism belongs to Legionella spp., the kit comprising:
(i) a hairpin probe having a stem-and-loop structure and comprising a microbead having a diameter of 1-50 μm, and an oligonucleotide having its 3′-end coupled to the microbead, wherein the oligonucleotide consists essentially of; from 5′ to 3′:
a Tag sequence hybridizable to a specific identification sequence of the target pathogenic microorganism,
an internal control sequence comprising at least four words, wherein each word consists of 4 nucleotides with a 75% AT-content, wherein the internal control sequence is operable to form the loop,
an anti-Tag sequence being a reverse complement of the Tag sequence so that the anti-Tag sequence and Tag sequence are operable to form the stem, and
a tail comprising at least two consecutive thymidine residues, and the oligonucleotide has any one of the following sequences:
(SEQ ID No: 1)
GCTTGTCTTCGAGTAAGGTTAAAAACGCATTAGCGTCATCATTCTAAACT
ACGCTAATGCGTTTTTAACCTTACTCGAAGACAAGCTTTTT,
(SEQ ID No: 2)
AAAGTCTGCTTAACGCCATTGCGAGCTATGTTCGCTCATCATTCATTACT
AGCGAACATAGCTCGCAATGGCGTTAAGCAGACTTTTTTTT,
(SEQ ID No: 3)
AAACCAGGAGTAGGATTCTCTTTTAGAAAAGAATTTCATTCATACTACTA
AAATTCTTTTCTAAAAGAGAATCCTACTCCTGGTTTTTTTT,
(SEQ ID No: 4)
CGCGTGCAGTCTTATCGAATTGTTCTTCCCGGGTATCATCTAACTAACTA
ATACCCGGGAAGAACAATTCGATAAGACTGCACGCGTTTTT,
(SEQ ID No: 5)
CTCCGGATCCACCGTTTCAGGGGCTTTATTTTCTATCATCTAACATTACT
ATAGAAAATAAAGCCCCTGAAACGGTGGATCCGGAGTTTTT,
(SEQ ID No: 6)
GCTTAAGTCATAGCGTTTCCATTCTTTATTAAAGATCATCTAACATTCTA
ATCTTTAATAAAGAATGGAAACGCTATGACTTAAGCTTTTT,
(SEQ ID No: 7)
TCAATTCTGGAAATGGTGTTAAACCCGGAAAATCGTCATACTAACTACTA
ACGATTTTCCGGGTTTAACACCATTTCCAGAATTGATTTTT,
and
(SEQ ID No: 8)
ACACTGATGTTCATTTGTTAGTCTCTTTTTACAATACTACTAATCATCAT
TATTGTAAAAAGAGACTAACAAATGAACATCAGTGTTTTTT;
(ii) an internal control conjugate, comprising,
a first quantum dot operable to produce a first luminescence having a first peak emission wavelength, and
an internal control probe conjugated to the first quantum dot, wherein the internal control probe has an internal control probe sequence complementary to the internal control sequence of the hairpin probe; and
(iii) a reporter conjugate, comprising,
a second quantum dot operable to produce a second luminescence having a second peak emission wavelength different from the first emission wavelength, and
a reporter probe conjugated to the second quantum dot, wherein the reporter probe has a reporter probe sequence complementary to the anti-Taq sequence.

10. The kit of the claim 9, wherein the first and second quantum dots respectively comprise a core selected from the group consisting of semiconductors, IIIB-VB semiconductors, and IVB-IVB semiconductors.

11. The kit of the claim 10, wherein the core is CdSe.

12. The kit of the claim 10, wherein the first and second quantum dots respectively further comprise a shell selected from ZnS and CdS.

13. The kit of claim 9, wherein the microbead comprises carboxylate-modified polystyrene.

14. The kit of claim 9, wherein the microbead has a diameter of 5-8.5 μm.

15. The kit of claim 9, wherein the Tag sequence and the anti-Tag sequence are respectively 18-100 nucleotides in length.

16. The kit of claim 9, wherein each of the words is selected from a. group consisting of CATT, CTAA, TCAT, and ACTA.

17. The kit of claim 9, wherein the tail is 2-8 nucleotides in length.

18. (canceled)

19. The kit of claim 9, further comprising a positive control probe which comprises a control microbead and the internal control sequence having its 3′-end directly coupled to the control microbead.

20. The kit of claim 9, further comprising a positive control probe which comprises a control microbead and the anti-Tag sequence having its 3′-end directly coupled to the control microbead.

21. The kit of claim 9, further comprising a negative control probe which comprises, from 5′ to 3′: the Tag sequence, the internal control sequence, anti-Tag sequence, and the tail.

22. The kit of claim 9, wherein the kit is operable for simultaneously detecting at least two target pathogenic microorganisms in a single reaction system and comprises,
at least two different hairpin probes, wherein for each of the hairpin probes:
the Tag sequence is hybridizable to the specific identification sequence of only one of the at least two target pathogenic microorganisms, and
the internal control sequence is uniquely assigned thereto;
at least two different internal control conjugates, each comprising:
the first quantum dot operable to produce the first luminescence having the first emission wavelength different from that of the remaining first quantum dot, and
the internal control probe sequence complementary to one of the internal control sequences; and
at least two different reporter conjugates, each comprising:
the second quantum dot operable to produce the second luminescence having the second emission wavelength different from the first emission wavelengths and that of the remaining second quantum dot, and
the reporter probe sequence complementary to the anti-Tag sequence of one of the hairpin probes.

23. A method for detecting a target pathogenic microorganism in a nucleic acid-containing sample, wherein the target pathogenic microorganism belongs to Legionella spp., the method comprising the steps of,
(i) mixing the nucleic acid-containing sample with a hairpin probe in a reaction system, wherein the hairpin probe has a stem-and-loop structure and comprises a microbead having a diameter of 1-50 μm, and an oligonucleotide having its 3′-end coupled to the microbead, wherein the oligonucleotide consists essentially of from 5′ to 3′:
a Tag sequence hybridizable to a specific identification sequence of the target pathogenic microorganism,
an internal control sequence comprising at least four words, wherein each word consists of 4 nucleotides with a 75% AT-content, wherein the internal control sequence is operable to form the loop,
an anti-Tag sequence being a reverse complement of the Tag sequence so that the anti-Tag sequence and Tag sequence are operable to form the stem, and
a tail comprising at least two consecutive thymidine residues, and the oligonucleotide has any one of the following sequences:
(SEQ ID No: 1)
GCTTGTCTTCGAGTAAGGTTAAAAACGCATTAGCGTCATCATTCTAAACT
ACGCTAATGCGTTTTTAACCTTACTCGAAGACAAGCTTTTT,
(SEQ ID No: 2)
AAAGTCTGCTTAACGCCATTGCGAGCTATGTTCGCTCATCATTCATTACT
AGCGAACATAGCTCGCAATGGCGTTAAGCAGACTTTTTTTT,
(SEQ ID No: 3)
AAACCAGGAGTAGGATTCTCTTTTAGAAAAGAATTTCATTCATACTACTA
AAATTCTTTTCTAAAAGAGAATCCTACTCCTGGTTTTTTTT,
(SEQ ID No: 4)
CGCGTGCAGTCTTATCGAATTGTTCTTCCCGGGTATCATCTAACTAACTA
ATACCCGGGAAGAACAATTCGATAAGACTGCACGCGTTTTT,
(SEQ ID No: 5)
CTCCGGATCCACCGTTTCAGGGGCTTTATTTTCTATCATCTAACATTACT
ATAGAAAATAAAGCCCCTGAAACGGTGGATCCGGAGTTTTT,
(SEQ ID No: 6)
GCTTAAGTCATAGCGTTTCCATTCTTTATTAAAGATCATCTAACATTCTA
ATCTTTAATAAAGAATGGAAACGCTATGACTTAAGCTTTTT,
(SEQ ID No: 7)
TCAATTCTGGAAATGGTGTTAAACCCGGAAAATCGTCATACTAACTACTA
ACGATTTTCCGGGTTTAACACCATTTCCAGAATTGATTTTT,
and
(SEQ ID No: 8)
ACACTGATGTTCATTTGTTAGTCTCTTTTTACAATACTACTAATCATCAT
TATTAGTAAAAAGAGACTAACAAATGAACATCAGTGTTTTTT;
(ii) denaturing the nucleic acid contained in the nucleic acid-containing sample and the hairpin probe at a denaturing temperature of 95-98° C., and then allowing a first hybridization to proceed at a first hybridizing temperature of 50-55° C. for 20 to 40 minutes such that the first hybridization between the hairpin, probe and the nucleic acid contained in the nucleic acid-containing sample is not complete;
(iii) adding excess amounts of an internal control conjugate and a reporter conjugate in the reaction system, and then allowing a second hybridization to proceed at a second hybridizing temperature of 50-55° C. for 30 to 120 minutes such that the hairpin probe is hybridizable to the internal control conjugate, reporter conjugate and/or the nucleic acid contained in the nucleic acid-containing sample, wherein,
the internal control conjugate comprises: a first quantum dot operable to produce a first luminescence having a first peak emission wavelength, and an internal control probe conjugated to the first quantum dot, wherein the internal control probe has an internal control probe sequence complementary to the internal control sequence of the hairpin probe, and
the reporter conjugate comprises: a second quantum dot operable to produce a second luminescence having a second peak emission wavelength different from the first emission wavelength, and a reporter probe conjugated to the second quantum dot, wherein the reporter probe has a reporter probe sequence complementary to the anti-Taq sequence;
(iv) removing unhybridized internal control conjugate and reporter conjugate from the reaction system;
(v) exciting the first and second quantum dots in the reaction system with an exciting wavelength; and
(vi) detecting the presence of the first luminescence and the second luminescence, wherein the presence of the second luminescence is indicative of the presence of the target pathogenic microorganism, and the number of events of the first luminescence is indicative of the amount of the target pathogenic microorganism in the sample.

24. The method of the claim 23, Wherein the step (vi) comprises performing flow cytometry analysis.

25. The method of claim 23, wherein the nucleic acid-containing sample is derived from an environmental sample or a biological sample.

26. The method of claim 25, wherein the nucleic acid-containing sample is used in the step (i) without prior nucleic acid amplification.
